(19) United States
(12) Patent Application Publication
(10) Pub. No.: US 2011/0165237 A1
(43) Pub. Date: Jul. 7, 2011

(54) Protein Hydrolysate Excipients

(75) Inventors: William Antonio Mark, Morgantown, WV (US); Lloyd Thomas Hall, Doswell, VA (US)

(73) Assignee: Wyeth, Madison, NJ (US)

(21) Appl. No.: 13/043,669

(22) Filed: Mar. 9, 2011

Related U.S. Application Data
(63) Continuation of application No. 11/354,974, filed on Feb. 16, 2006.

(51) Int. Cl. A61K 31/192; A61K 9/28; A61P 29/00

(52) U.S. Cl. 424/465; 514/570; 427/2.14

(57) ABSTRACT

A pharmaceutical composition comprising an effective amount of a pharmaceutical active and up to about 99.8% wt/wt water soluble protein hydrolysate to total weight of composition is provided. Whey protein hydrolysate is exemplary of a suitable soluble protein hydrolysate. A method for preparing such a composition is also provided.

### FIELD OF THE INVENTION

The present invention relates to pharmaceutical compositions and more particularly to pharmaceutical compositions for delivery of a pharmaceutical active and a method for preparing such compositions.

### BACKGROUND OF THE INVENTION

Typically, when therapeutic agents are administered to humans in a dosage form, the therapeutic active agent(s) (e.g. active agent) is administered in a composition that facilitates delivery and/or bioavailability of the active agent. The dosing of a number of active agents present particular challenges and it is desirable to craft the composition of the dosage form to overcome the challenges. For example, hydrophobic therapeutic active agents, which have poor solubility in aqueous solutions, present problems for internal administration to humans as the human biological system is aqueous based. For effective administration, a therapeutically effective amount of the hydrophobic active agent must be delivered to the desired absorption site in an absorbable form. Further, any solvents or excipients used to transport the hydrophobic agent and/or to maintain or create the absorbable form of the hydrophobic active agent need to be physiologically compatible.

Formulations of therapeutic active agents include solid and liquid compositions. In the case of active agents with low water solubility, specialized liquid systems have been used. Such liquid compositions may employ, for example, an oil-in-water emulsion, a microemulsion, a solution of micelles, liposomes or multi-lamellar carrier particles to facilitate delivery.

A number of specific methods and compositions for delivery of therapeutic actives in solid form have been set forth. For example, WO 02/080881 discloses a process for making protein particles for delivery of a bioactive molecule by utilizing denatured protein. The denatured protein is used to form an emulsion, and the emulsion is treated with salt to form particles. Whey protein is one of a number of proteins that are listed as useful in the practice of the invention. Particles are defined as having a size range from 5 micrometers to 8 millimeters in diameter in WO 02/080881.

U.S. Pat. No. 4,670,251 (the “'251 Patent”) is directed to a microcrystalline solid product derived from a dairy whey lactose permeate which may be used as a binder for solid pharmaceutical compositions suitable for oral or rectal administration. The composition of the '251 Patent is rich in lactose.

Accordingly, there is a need for a simple effective system for delivery of pharmaceutical active agents in solid dosage forms.

### SUMMARY OF THE INVENTION

The pharmaceutical composition described herein comprises an effective amount of a pharmaceutical active and up to about 99.8% wt/wt water soluble protein hydrolysate to total weight of the composition. The pharmaceutical composition may be a dosage form selected from a mini-capsule, a capsule, a tablet, a troche, a lozenge a minitablet, a suspension, an ovule, a suppository, a wafer; a chewable tablet, an effervescent tablet, a caplet, a buccal or sublingual solid, a granulation, a microsphere, a film, a sprinkle, a pellet, a bead, a pill, a powder, a triturate, a platelet, a strip, a sachet, a lyophilized cake, a foam and combinations thereof.

The pharmaceutical active may be selected from analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, diuretics, expectorants, gastrointestinal agents, ionizable hydrophobic active agents, migraine preparations, motion sickness products, mucolytics, muscle relaxants, non-steroidal anti-inflammatory drugs (NSAIDs), nutritional supplements, COX-2 inhibitors, osteoporosis preparations, polydimethylsiloxanes, respiratory agents, sleep-aids, urinary tract agents, antipyretics and mixtures thereof, for example.

In an exemplary embodiment the pharmaceutical composition comprises an effective amount of a hydrophobic pharmaceutical active and whey protein hydrolysate. The whey protein hydrolysate may comprise up to about 99.8% wt/wt soluble protein hydrolysate to total weight of the composition of the pharmaceutical composition and typically comprises about 0.01 to 60% wt/wt of soluble protein hydrolysate to total weight of the composition.

A method of preparing a pharmaceutical composition is also provided. The method comprises providing a soluble protein hydrolysate, providing an effective amount of at least one pharmaceutical active, and combining the soluble protein hydrolysate and the effective amount of the at least one pharmaceutical active. The method of combining the soluble protein hydrolysate and the effective amount of at least one pharmaceutical active may be selected from dry mixing, solvent mixing, agglomerating, air suspension chilling, air suspension drying, balling, coacervations, coating, compressing, cryopelletization, encapsulation, extrusion, wet granulation, dry granulation, homogenization, inclusion complexation, lyophilization, melting, microencapsulation, molding, pan coating, precipitation, solvent dehydration, sonication, spheronization, spray chilling, spray congealing, spray drying, melting and cooling with recrystallization, and combinations thereof, for example.

Optionally, the dosage form may be coated with a film coat, modified film coat, sugar coat, compression coat, or laminates applied by various means.

### BRIEF DESCRIPTION OF THE FIGURES

FIG. 1 is a graph showing dissolution of an exemplary embodiment of the invention as compared to a reference composition.

FIG. 2 is a graph showing dissolution of an exemplary embodiment of the invention as compared to a reference composition.

FIG. 3 is a graph showing dissolution of an exemplary embodiment of the invention as compared to a reference composition.

FIG. 4 is a graph showing dissolution of two exemplary embodiments of the invention as compared to a reference composition.

FIG. 5 is a graph showing dissolution of an exemplary embodiment of the invention as compared to a reference composition.

### DETAILED DESCRIPTION OF THE INVENTION

The inventors have discovered a simple effective solid dosage system utilizing soluble hydrolyzed protein (i.e. soluble protein hydrolysate). The soluble protein hydrolysate serves as an excipient which may perform one or multiple excipient functions. The soluble protein hydrolysate may function as a solubilizer, a binder, a buffer, a chelating agent, a complexing agent, a surfactant, a modified release agent, a diluent, a filler, or dispersant, or some combination thereof. As used herein soluble protein hydrolysate means a soluble protein hydrolysate derived from a non-gelatin protein. Globular proteins, plant proteins, and proteins from protista, monera and fungi are exemplary of suitable proteins from which the soluble protein hydrolysate may be formed.

The use of soluble whey protein hydrolysate as described in the examples herein is exemplary. In some embodiments whey protein hydrolysate is particularly useful as a solubilizer and/or dispersant and/or wetting agent (e.g., as an agent for enhancing dissolution of a solid preparation). In some embodiments hydrolyzed whey protein may be used as the sole solubilizer. Whey protein hydrolysate may also function as a binder, buffer, chelating agent, diluent, antioxidant, or dispersant. Whey protein hydrolysate may, in some embodiments, perform a combination of two or more of these functions. Thus, in some embodiments, not only is whey protein hydrolysate a useful excipient but also the number and/or amounts of ingredients in a pharmaceutical preparation may be reduced by replacement of conventional excipients with soluble hydrolyzed protein which may perform multiple functions. Alternatively, whey protein hydrolysate may be used in combination with other solublizers, binders, bufferants, chelating agents, diluents and dispersants. In embodiments using whey protein hydrolysate in combination with other excipients, the whey protein hydrolysate may provide a particular benefit, such as for example, enhancing dispersion and/or perform one or more functions which facilitate the reduction of the amounts of other excipients. Accordingly, the use of soluble hydrolyzed protein as an excipient may reduce manufacturing costs by replacing one or more expensive additives with soluble hydrolyzed protein and/or reducing the amount of additives needed.

The solid dosage forms described herein comprise soluble protein hydrolysate in which the hydrolysate is soluble in aqueous solution and at least one therapeutic agent (also referred to herein as “active agent” or “pharmaceutical active” or “active”). The composition may optionally comprise other excipients. Dosage forms may be prepared by combining the soluble protein hydrolysate with one or more active agents and optionally with one or more additional excipients. Mixing techniques such as dry mixing, including ordered and/or high shear mixing, solvent mixing, agglomerating, air suspension chilling, air suspension drying, balling, coacervation, coating, compressing, cryopelletization, encapsulation, homogenization, inclusion complexation, lyophilization, molding, melting, pan coating, precipitation, solvent dehydration, sonication, spheronization, spray congealing, spray drying, melting and cooling with recrystallization, precipitation, extrusion, foaming or granulation or combinations thereof may be employed in forming the composition, for example. Once combined, the resulting composition may be used in the form of a powder, sachet, sprinkle granulation, microsphere, pellet, lyophilized cake, filled into a capsule or mini-capsule, formed into a tablet, caplet, film, bead, foam or combination thereof, for example. Alternatively, the composition may be used in a liquid form.

The soluble protein hydrolysate may comprise up to about 99.8% wt/wt of the dosage form (weight of soluble protein hydrolysate to total weight of the composition). However, typically lesser amounts of soluble protein hydrolysate are used, such as, for example, about 0.01% wt/wt to about 60% wt/wt soluble protein hydrolysate to total weight of the composition. In designing dosage forms, such as dosage forms in which the soluble protein hydrolysate facilitates dissolution, for example, it may be desirable to consider the proportion of weight of soluble protein hydrolysate to weight of active plus soluble protein hydrolysate. Accordingly, unless otherwise indicated, percentages do not refer to the entire composition but rather to the relative proportion of soluble protein hydrolysate to the active agent(s) plus soluble protein hydrolysate. The amount of soluble protein hydrolysate used may impact the dispersion rate of the pharmaceutical active. In one exemplary embodiment comprising hydrolyzed whey protein and ibuprofen, amounts of hydrolyzed whey protein up to about 20% wt/wt typically enhanced solubilization/dispersion of the ibuprofen while amounts of whey protein greater than about 20% wt/wt modulated and/or slowed solubilization/dispersion. The enhanced dispersion or modulation and/or slowed solubilization/dispersion are determined as compared to a composition similar in composition except for lacking the soluble protein hydrolysate.

The inventors believe, without wishing to be bound to the theory, that the soluble protein hydrolysate may either enhance or slow dispersion of a pharmaceutical active depending on the amount of soluble protein hydrolysate used. A whey protein hydrolysate/ibuprofen embodiment is exemplary, and the specific amount of hydrolyzed protein needed to either enhance dispersion or slow dispersion depends on the physical and/or chemical properties of the pharmaceutical active, the specific chemical structure of the soluble protein hydrolysate and the nature of any other excipients used. Similarly, for the hydrolyzed whey protein/ibuprofen embodiment the percentage of hydrolyzed whey protein needed to enhance dissolution may be less than 20% wt/wt or the amount of hydrolyzed whey protein to slow dissolution may be greater than 20% greater wt/wt in the presence of other excipients and/or other active agents. The amount of soluble hydrolyzed protein needed to achieve the desired effect can be determined experimentally by using dissolution experiments, for example.

Whey protein hydrolysates, for example, may be derived by hydrolysis of whey. Hydrolysis with acid, base or by enzymatic means are exemplary of methods for obtaining whey protein hydrolysate. The choice of hydrolysis methods impacts the properties of the hydrolysate peptides as different hydrolysis methods and/or agents cleave proteins differently. For example, the enzyme trypsin cleaves proteins to reveal arginine and lysine residues, and chymotrypsin cleaves carboxyl links of hydrophobic amino acids. Hence, use of trypsin would create peptide fragments with lysine and arginine terminus amino acids and chymotrypsin would create peptide fragments with hydrophobic amino acid residues. Optionally, multiple hydrolysis steps may be performed using different hydrolysis methods and/or agents to customize the properties of the hydrolysate. Alternatively, a protein sample may be divided into aliquots and different hydrolysis methods may be applied to each of the separate aliquots of protein. The resulting hydrolysates may be combined in a selected proportion to be used as an excipient with a customized distribution of peptide end groups. Similarly, different protein samples may be hydrolyzed and combined and used as an excipient to give a customized distribution of peptide fragments. The degree of hydrolysis and positions of cleavage may be selected to impart one or more specific properties, such as for example, enhancing solubilization of a particular therapeutic agent and/or modulating buffering capacity, for example. Accordingly, considerable flexibility is provided for optimizing and/or selecting particular types of peptides with particular physical and/or chemical properties.

A whey protein hydrolysate suitable for use as an excipient in pharmaceutical preparation with hydrophobic active agents such as ibuprofen, for example, is commercially available. Namely, whey protein hydrolysate made by Davisco Foods International 12100 West 78^{th} Street, Eden Prairie, Minn. 55344 and marketed under the name Biozate for use as a nutritive component of a nutritional supplement is suitable for use as an excipient in ibuprofen compositions, for example. This hydrolysate is substantially free of lactose.

Whey protein hydrolysate, as discussed in detail in the following description, can impart desirable properties in a pharmaceutical composition, such as, for example, enhanced dispersion of an active agent or other modulation of release of an active agent and/or perform one or more typical excipient functions. Whey protein hydrolysate may provide a special specific benefit and/or replace other excipients or reduce the amount of other excipients needed.

Whey protein hydrolysate is exemplary, and it should be understood that other non-gelatin, soluble protein hydrolysates such as, for example, hydrolysates of milk protein, casein, soy protein, wheat gluten, corn gluten, yeast protein, egg protein and mixtures thereof, may be likewise suitable in the practice of the invention if the hydrolyzed material (e.g. the hydrolysate) is soluble in an aqueous solution. Accordingly, the term soluble protein hydrolysate means peptide(s) or peptide derivative(s) obtained from the hydrolysis of a protein wherein the peptide(s) or peptide derivative(s) are soluble in aqueous solution. It is not required that the original protein be soluble in aqueous solution, but rather that the fragments obtained or some portion of the fragments obtained from hydrolysis of the original protein be soluble in aqueous solution. Soluble protein hydrolysates other than whey protein hydrolysate may likewise perform excipient functions such as acting as solubilizers, wetting agents, binders, buffering agents, chelating agents, diluent, fillers, dispersants or some combination thereof.

Soluble protein hydrolysates such as whey protein hydrolysates may perform the function of a solubilizer in a pharmaceutical composition. Solubilizers are additives used to increase the solubility of the pharmaceutical active, or other composition components in the pharmaceutical preparation. In some embodiments, whey protein hydrolysates may function to facilitate solubilization by acting as a wetting agent. The soluble protein hydrolysate may serve as the solubilizer alone or in combination with one or more known solublizers. Known suitable pharmaceutical solublizers include, but are not limited to: alcohols and polyols, such as, ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinyl alcohol, hydroxypropyl methylcellulose and other cellulose derivatives; cyclodextrins and cyclodextrin derivatives; ethers of polyethylene glycol, polyvinyl pyrrolidine (PVP) having an average molecular weight of about 200 to about 6000; amides such a 2-pyrrolidone, 2 piperidone, caprolactam, N-alkylpyrrolidione, N-hydroxyalkylpyrrolidine, N-alkylpiperidione, N-alkylcaprolactam, dimethylacetamide, and polyvinylpyrrolidone; esters, such as ethyl propionate, tributylcitrate, acetyltriethylcitrate, acetyl tributyl citrate, triethylcitrate, ethyl oleate, ethyl caprylate, ethyl butyrate, triacetin, propylene glycol monoacetate, propylene glycol diacetate, caprolactone, and isomers thereof and, valerolactone, and isomers thereof, and butyrolactone and isomers thereof; mono-, di-, and tri-fatty acid esters of glycerol, esters of sorbitol and sorbitans, water and mixtures thereof. The amount of a particular solubilizer and or total amount of solublizers used in the composition is limited to a bio-acceptable amount which is readily determined by one skilled in the art. Typically, when whey protein hydrolysate, for example, is used in combination with one or more other solublizers, the amount of non-hydrolyzed whey protein solubilizer is reduced as compared to the amount that would be used in the absence of whey protein hydrolysate.

Soluble protein hydrolysate, such a whey protein hydrolysate, may be used as a binder. A binder is an agent that imparts cohesive properties to powdered or particulate materials through particle-to-particle binding. Binders which may be used in combination with soluble protein hydrolysate include, but are not limited to: dry starch, dry sugars; polyvinyl pyrrolidine; starch paste; celluloses; bentonite; sucrose; polymeric cellulose derivatives, such as carboxymethylcellulose; hydroxypropylcellulose, and hydroxpropylmethylcellulose; sugar syrups; corn syrup; water soluble polysaccharides, such as acacia, tragacanth, guar, and alginates, gelatin, agar, sucrose, dextrose, polyethylene glycol, (PEG), vinyl copolymers, pregelatinized starch, sorbitol and glucose. Soluble protein hydrolysate may be used as a sole binder or may be used in combination with one or more of the conventional binders. Typically, when whey protein hydrolysate, for example, is used as a binder in combination with a conventional binder, the amount of conventional binder can be reduced with respect to the amount that would be used in the absence of hydrolyzed whey protein.

Due to the presence of some amino acid functional groups in soluble protein hydrolysate, the soluble protein hydrolysate has substantial buffering capacity. The amount of buffering capacity may be modulated by selection of parameters (e.g. agents) associated with the hydrolysis process, for example. In some embodiments, the buffering capacity of hydrolyzed protein may be sufficient to provide the desired buffering properties for the pharmaceutical composition. In other embodiments, it may be desirable to use soluble protein hydrolysate in combination with one or more other known bufferants. Exemplary bufferants include, but are not limited to, hydrochloric acid, hydrobromic acid, hydroiodic acid, sulfuric acid, nitric acid, boric acid, phosphoric acid, acetic acid, acrylic acid, adipic acid, alginic acid, alkane sulfonic acid, amino acids, ascorbic acid, benzoic acid, boric acid, butyric acid, carbonic acid, citric acid, fatty acid, formic acid, fumaric acid, gluconic acid, hydroquinosulfonic acid, isoascorbic acid, lactic acid, maleic acid, methane sulfonic acid, oxalic acid, para-bromophenylsulfonic acid, propionic acid, p-toluenesulfonic acid, salicylic acid, steric acid, succinic acid, tannic acid, tartaric acid, thioglycolic acid, toluenesulfonic acid, and uric acid and their conjugate salts. Pharmaceutical acceptable bases such as amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopopropanolamine, or a salt of a pharmaceutically acceptable cation and acetic acid, ascorbic acid, adipic acid, alginic acid, alkanesulfonic acid, amino acid, ascorbic acid, benzoic acid, boric acid, butyric acid, carbonic acid, citric acid, a fatty acid, formic acid, fumaric acid, gluconic acid, hydroquinonesulfsonic acid, isoascorbic acid, lactic acid, maleic acid, methanesulfonic acid, oxalic acid, para-bromophenylsulfonic acid, propionic acid, p-toluenesulfonic acid, salicylic acid, steric acid, succinic acid, tannic acid, tartaric acid, thioglycolic acid, toluesulfonic acid. Amphoteric compounds such as amino acids and multivalent cations may also act as buffers.

In some embodiments, soluble protein hydrolysate may function as a chelating agent. For example, whey protein hydrolysate may chealate ions such as calcium ions, iron ions, and the like.

A filler or diluent is an ingredient used to add bulk to a solid dosage form. Typically, filler adds bulk which facilitates handling the composition and, in many instances, fillers do not contribute substantially to the chemical properties of the composition. Soluble protein hydrolysate may function as a diluent or filler. Soluble protein hydrolysate may be used in place of other diluents or fillers or in combination with other diluents and fillers. Exemplary diluents and fillers that may be used in combination with soluble protein hydrolysate include, but are not limited to lactose, mannitol, talc, magnesium stereate, sodium chloride, potassium chloride, citric acid, spray-dried lactose, hydrolyzed starches, directly compressable starch, microcrystalline cellulose, cellulosics, sorbitol, sucrose, sucrose based materials, calcium sulfate, dibasic calcium phosphate, and dextrose.

Soluble protein hydrolysate may, in some embodiments, serve as a dispersant. The use of whey protein hydrolysate as a dispersant, for example, may reduce the need for disintegrants or superdisintregrants in a pharmaceutical composition in some embodiments. Common disintegrants or superdisintegrants include, but are not limited to croscarmellose sodium, starch, starch derivatives, clay, gum, cellulose, cellulose derivatives, alginates, crosslinked polyvinylpyrrolidone sodium starch glycolate and micro-crystalline cellulose. Whey protein hydrolysate, for example, may serve as the sole dispersant or in combination with other disintegrants or superdisintegrants.

Soluble protein hydrolysate may be used in formulations with any type of pharmaceutical actives. Exemplary suitable pharmaceutical activities include but are not limited to analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, diuretics, expectorants, gastrointestinal agents, ionizable hydrophobic active agents, migraine preparations, motion sickness products, mucolytics, muscle relaxants, non-steroidal anti-inflammatory drugs (NSAIDs), nutritional supplements, Cox-2 inhibitors, osteoporosis preparations, polydimethylsiloxanes, respiratory agents, sleep-aids, urinary tract agents, antipyretics and mixtures thereof. Soluble protein hydrolysates may be particularly useful for formulation of therapeutic agents that present challenges, for example, whey protein hydrolysate is particularly useful as an excipient for formulations comprising ionizable hydrophobic therapeutic agents.

Ionizable hydrophobic therapeutic agents are compounds with little water solubility in un-ionized form. Water solubilities (i.e., water solubility of the un-ionized form) for the ionizable hydrophobic therapeutic agents is typically less than about 1% by weight (e.g. weight of hydrophobic therapeutic agent to weight of water), and may be less than about 0.1% or 0.01% by weight. A wide variety of ionizable hydrophobic therapeutic agents can be effectively incorporated in and delivered by the pharmaceutical compositions comprising a soluble protein hydrolysate, such as whey protein hydrolysate, for example.

An ionizable hydrophobic therapeutic agent is characterized by the presence of at least one ionizable functional group. Ionizable functional groups can be acidic groups, or basic groups, with “acidic” and “basic” referring to acidic or basic behavior in a Bronsted-Lowry or Lewis acid/base sense. The terms “acid” and “base” as used herein refer to the ability of a functional group to act as a Bronsted-Lowry acid or Lewis acid, or as a Bronsted-Lowry base or Lewis base, in the presence of an appropriate ionizing agent. For simplicity, the acidic and basic properties of functional groups; ionizing agents, and neutralizing agents are described herein with particular reference to Bronsted-Lowry properties, but the corresponding Lewis acid/base properties are also included within the scope of these terms.

This usage should be contrasted with the terminology typically used in describing whether a compound is “acidic” or “basic” based on the pKₐ of the compound in deionized water. For example, the equivalent pKₐ of a functional group need not be less than 7 to be considered “acidic”, since even functional groups with a large pKₐ can be “acidic” if they can be deprotonated by a strong base. Similarly, a functional group with an equivalent pKₐ of less than 7 may still be considered “basic” if it can be protonated by a stronger acid. Thus, it is the ability of a particular functional group to be ionized (protonated or deprotonated) by a suitable ionizing agent (acid or base) that determines whether a functional group is acidic or basic, rather than the particular pKₐ associated with that group or with the compound as a whole. Accordingly, acidic functional groups are those groups that can be deprotonated by a suitable base to yield the corresponding anionic group (the conjugate base), or groups that can accept an electron pair. Basic functional groups are those groups that can be protonated by a suitable acid to yield the corresponding cationic group (the conjugate acid), or can donate an electron pair.

Ionizable hydrophobic therapeutic agents contain at least one ionizable functional group. Of course, many suitable therapeutic agents contain a plurality of such groups, and a single therapeutic agent may contain one or more acidic functional groups as well as one or more basic functional groups. Such therapeutic agents are also within the scope of the present invention.

Acidic functional groups include, but are not limited to, carboxylic acids, imidazolidinediones, thiazolidinediones, pyrimidinetriones, hydroxyheteroaromatics, phenols, phosphoric acids, sulfuric acids, sulfonic acids, sulfonamides, aminosulfones, sulfonylureas, tetrazoles and thiols, for example.

In order to avoid particularly cumbersome terminology, the functional groups, whether acidic or basic, are referred to by naming the corresponding free compound. For example, referring to a functional group, the term “aminosulfone” is used, rather than the more technically precise term “aminosulfonyl”, such designation is common in the art.

Basic functional groups include, but are not limited to, aliphatic amines, aromatic amines, C-substituted aromatic amines, N-substituted aromatic amines, heterocyclic amines, C-substituted heterocyclic amines and N-substituted heterocyclic amines, for example.

Examples of aromatic amines and substituted aromatic amines include, but are not limited to, aniline, N-methylaniline and p-toluidine.

Examples of heterocyclic and substituted heterocyclic amines include, but are not limited to, pyrrole, pyrazole, imidazole, indole, pyridine, pyridazine, pyrimidine, quinoline, piperidine, pyrrolidine, morpholine, thiazole, purine and triazole.

Specific examples of ionizable hydrophobic therapeutic agents having at least one ionizable acidic functional group include, but are not limited to: acetazolamide, acetohexamide, acrivastine, alatrofloxacin, albuterol, alclofenac, aloxiprin, alprostadil, amodiaquine, amphotericin, amylobarbital, aspirin, atorvastatin, atovaquone, baclofen, barbital, benazepril, bezafibrate, bromfenac, bumetanide, butobarbital, candesartan, capsaicin, captopril, cefazolin, celecoxib, cephadrine, cephalexin, cerivastatin, cetrizine, chlorambucil, chlorothiazide, chlorpropamide, chlorthalidone, cinoxacin, ciprofloxacin, clinofibrate, cloxacillin, cromoglicate, cromolyn, dantrolene, dichlorophen, diclofenac, dicloxacillin, dicumarol, diflunisal, dimenhydrinate, divalproex, docusate, dronabinol, enoximone, enalapril, enoxacin, enrofloxacin, epalrestat, eposartan, essential fatty acids, estramustine, ethacrynic acid, ethotoin, etodolac, etoposide, fenbufen, fenoprofen, fexofenadine, fluconazole, flurbiprofen, fluvastatin, fosinopril, fosphenytoin, fumagillin, furosemide, gabapentin, gemfibrozil, gliclazide, glipizide, glybenclamide, glyburide, glimepiride, grepafloxacin, ibufenac, ibuprofen, imipenem, indomethacin, irbesartan, isotretinoin, ketoprofen, ketorolac, lamotrigine, levofloxacin, levothyroxine, lisinopril, lomefloxacin, losartan, lovastatin, meclofenamic acid, mefenamic acid, mesalamine, methotrexate, metolazone, montelukast, nalidixic acid, naproxen, natamycin, nimesulide, nitrofurantoin, non-essential fatty acids, norfloxacin, nystatin, ofloxacin, oxacillin, oxaprozin, oxyphenbutazone, penicillins, pentobarbital, perfloxacin, phenobarbital, phenyloin, pioglitazone, piroxicam, pramipexol, pranlukast, pravastatin, probenecid, probucol, propofol, propylthiouracil, quinapril, rabeprazole, repaglinide, rifampin, rifapentine, sparfloxacin, sulfabenzamide, sulfacetamide, sulfadiazine, sulfadoxine, sulfamerazine, sulfamethoxazole, sulfafurazole, sulfapyridine, sulfasalazine, sulindac, sulphasalazine, sulthiame, telmisartan, teniposide, terbutaline, tetrahydrocannabinol, tirofiban, tolazamide, tolbutamide, tolcapone, tolmetin, tretinoin, troglitazone, trovafloxacin, undecenoic acid, ursodeoxycholic acid, valproic acid, valsartan, vancomycin, verteporfin, vigabatrin, and zafirlukast.

Among the above-listed hydrophobic therapeutic agents having at least one acidic functional group, preferred hydrophobic therapeutic agents are: alclofenac, aspirin, atorvastatin, atovaquone, benazepril, bromfenac, celecoxib, cromoglicate, cromolyn, diclofenac, dronabinol, etodolac, fexofenadine, flurbiprofen, glimepiride, ibufenac, ibuprofen, isotretinoin, ketoprofen, ketorolac, levothyroxine, naproxen, non-essential fatty acids, oxaprozin, phenyloin, pioglitazone, rabeprazole, repaglinide, teniposide, tetrahydrocannabinol, tolmetin, tretinoin, troglitazone, and trovafloxacin.

Specific examples of suitable hydrophobic therapeutic agents having at least one ionizable basic functional group include, but are not limited to: abacavir, acebutolol, acrivastine, alatrofloxacin, albuterol, albendazole, alprazolam, alprenolol, amantadine, amiloride, aminoglutethimide, amiodarone, amitriptyline, amlodipine, amodiaquine, amoxapine, amphetamine, amphotericin, amprenavir, amrinone, amsacrine, astemizole, atenolol, atropine, azathioprine, azelastine, azithromycin, baclofen, benethamine, benidipine, benzhexol, benznidazole, benztropine, biperiden, bisacodyl, bisanthrene, bromazepam, bromocriptine, bromperidol, brompheniramine, brotizolam, bupropion, butenafine, butoconazole, cambendazole, camptothecin, carbinoxamine, cephadrine, cephalexin, cetrizine, cinnarizine, chlorambucil, chlorpheniramine, chlorproguanil, chlordiazepoxide, chlorpromazine, chlorprothixene, chloroquine, cimetidine, ciprofloxacin, cisapride, citalopram, clarithromycin, clemastine, clemizole, clenbuterol, clofazimine, clomiphene, clonazepam, clopidogrel, clozapine, clotiazepam, clotrimazole, codeine, cyclizine, cyproheptadine, dacarbazine, darodipine, decoquinate, delavirdine, demeclo-cycline, dexamphetamine, dexchlorpheniramine, dexfenfluramine, diamorphine, diazepam, diethylpropion, dihydrocodeine, dihydroergotamine, diltiazem, dimenhydrinate, diphenhydramine, diphenoxylate, diphenyl-imidazole, diphenylpyraline, dipyridamole, dirithromycin, disopyramide, dolasetron, domperidone, donepezil, doxazosin, doxycycline, droperidol, econazole, efavirenz, ellipticine, enalapril, enoxacin, enrofloxacin, eperisone, ephedrine, ergotamine, erythromycin, ethambutol, ethionamide, ethopropazine, etoperidone, famotidine, felodipine, fenbendazole, fenfluramine, fenoldopam, fentanyl, fexofenadine, flecainide, flucytosine, flunarizine, flunitrazepam, fluopromazine, fluoxetine, fluphenthixol, fluphenthixol decanoate, fluphenazine, fluphenazine decanoate, flurazepam, flurithromycin, frovatriptan, gabapentin, granisetron, grepafloxacin, guanabenz, halofantrine, haloperidol, hyoscyamine, imipenem, indinavir, irinotecan, isoxazole, isradipine, itraconazole, ketoconazole, ketotifen, labetalol, lamivudine, lanosprazole, leflunomide, levofloxacin, lisinopril, lomefloxacin, loperamide, loratadine, lorazepam, lormetazepam, lysuride, mepacrine, maprotiline, mazindol, mebendazole, meclizine, medazepam, mefloquine, melonicam, meptazinol, mercaptopurine, mesalamine, mesoridazine, metformin, methadone, methaqualone, methylphenidate, methylphenobarbital, methysergide, metoclopramide, metoprolol, metronidazole, mianserin, miconazole, midazolam, miglitol, minoxidil, mitomycins, mitoxantrone, molindone, montelukast, morphine, moxifloxacin, nadolol, nalbuphine, naratriptan, natamycin, nefazodone, nelfinavir, nevirapine, nicardipine, nicotine, nifedipine, nimodipine, nimorazole, nisoldipine, nitrazepam, nitrofurazone, nizatidine, norfloxacin, nortriptyline, nystatin, ofloxacin, olanzapine, omeprazole, ondansetron, omidazole, oxamniquine, oxantel, oxatomide, oxazepam, oxfendazole, oxiconazole, oxprenolol, oxybutynin, oxyphencyclimine, paroxetine, pentazocine, pentoxifylline, perchlorperazine, perfloxacin, perphenazine, phenbenzamine, pheniramine, phenoxybenzamine, phentermine, physostigmine, pimozide, pindolol, pizotifen, pramipexol, pranlukast, praziquantel, prazosin, procarbazine, prochlorperazine, proguanil, propranolol, pseudoephedrine, pyrantel, pyrimethamine, quetiapine, quinidine, quinine, raloxifene, ranitidine, remifentanil, repaglinide, reserpine, ricobendazole, rifabutin, rifampin, rifapentine, rimantadine, risperidone, ritonavir, rizatriptan, ropinirole, rosiglitazone, roxatidine, roxithromycin, salbutamol, saquinavir, selegiline, sertraline, sibutramine, sildenafil, sparfloxacin, spiramycins, stavudine, sulconazole, sulphasalazine, sulpiride, sumatriptan, tacrine, tamoxifen, tamsulosin, temazepam, terazosin, terbinafine, terbutaline, terconazole, terfenadine, tetramisole, thiabendazole, thioguanine, thioridazine, tiagabine, ticlopidine, timolol, tinidazole, tioconazole, tirofiban, tizanidine, tolterodine, topotecan, toremifene, tramadol, trazodone, triamterene, triazolam, trifluoperazine, trimethoprim, trimipramine, tromethamine, tropicamide, trovafloxacin, vancomycin, venlafaxine, vigabatrin, vinblastine, vincristine, vinorelbine, vitamin K₁, vitamin K₂, vitamin K₅, vitamin K₆, vitamin K₇, zafirlukast, zolmitriptan, zolpidem and zopiclone.

Among the above-listed ionizable hydrophobic therapeutic agents having at least one ionizable basic functional group, preferred hydrophobic therapeutic agents are: amlodipine, astemizole, brompheniramine, bupropion, carbinoxamine, cetrizine, cimetidine, cisapride, clemastine, clemizole, dihydroergotamine, diphenhydramine, diphenylimidazole, diphenylpyraline, domperidone, famotidine, fexofenadine, frovatriptan, granisetron, itraconazole, ketoconazole, ketotifen, lanosprazole, leflunomide, loperamide, loratadine, methysergide, miglitol, montelukast, naratriptan, nizatidine, omeprazole, ondansetron, phenbenzamine, pseudoephedrine, raloxifene, repaglinide, rifabutin, rimantadine, ritonavir, rizatriptan, rosiglitazone, roxatidine, saquinavir, sibutramine, sildenafil, sumatriptan, tamsulosin, terbinafine, tizanidine, tramadol, trovafloxacin, vitamin K₁, vitamin K₂, vitamin K₅, vitamin K₆, vitamin K₇, zafirlukast, zolmitriptan and zolpidem.

Also included within the scope of the invention are pharmaceutically equivalent derivatives and/or analogs of the ionizable hydrophobic therapeutic agents. Such equivalents include but are not limited to both ionized and unionized forms, salts, esters, alkyl, acyl derivatives and combinations thereof.

In particular, salts of ionizable hydrophobic therapeutic agents are suitable for use in the present invention. In some embodiments use of a mixture of ionized hydrophobic therapeutic agent and a salt or salts of the hydrophobic therapeutic agent may be desirable.

It should be appreciated that this listing of ionizable hydrophobic therapeutic agents is merely illustrative. Indeed, a particular feature of the compositions of the present invention is the ability of the present compositions to facilitate solubilization and/or delivery of a broad range of ionizable hydrophobic therapeutic agents, regardless of therapeutic class. Of course, mixtures of ionizable hydrophobic therapeutic agents may also be used if desired.

Although the use of soluble hydrolyzed protein with hydrophobic pharmaceutical activities may be particularly beneficial in some embodiments, the use of soluble protein hydrolysate to perform one or more excipient function in compositions with one or more types of pharmaceutical activities other than hydrophobic pharmaceutical activities or with combinations of pharmaceutical actives may be desirable as well. Accordingly, use of soluble hydrolyzed protein such as whey protein hydrolysate as an excipient in combination with a pharmaceutical active includes use with ionizable hydrophobic pharmaceutical activities and other types of pharmaceutical activities or combinations thereof.

The composition of the invention can be processed by dry mixing, solvent mixing, agglomerating, air suspension chilling, air suspension drying, balling, coacervations, coating, compressing, cryopelletization, encapsulation, extrusion, wet granulation, dry granulation, homogenization, inclusion complexation, lyophilization, melting, microencapsulation, molding, pan coating, precipitation, solvent dehydration, sonication, spheronization, spray chilling, spray congealing, spray drying, melting and cooling with recrystallization or other processes known in the art.

The composition can be provided in the form of a mini-capsule, a capsule, a tablet, a caplet a troche, a lozenge, a minitablet, a temporary or permanent suspension, an ovule, a suppository, a wafer, a chewable tablet, an effervescent tablet, a buccal or sublingual solid, a granulation, a film, a sprinkle, a pellet, a bead, a pill, a powder, a triturate, a platelet, a strip, a sachet, a lyophilized cake, a foam and combinations thereof. Typically the composition is formulated for oral delivery. However in some embodiments delivery may be nasal, buccal, ocular, urethral, transmucosal, vaginal, topical or rectal.

The dosage unit of the composition and/or particles of the composition may be coated with one or more coatings. Coatings may include, for example, enteric coatings, seal coatings such as HPMC and/or ethyl cellulose in combination or Eudragit E100, for example, film coatings, modified film coatings, barrier coatings, compression coatings, enzyme degradable coatings, sugar coatings. Multiple coatings and/or laminates and/or layers of coatings may be used in some embodiments.

The coating may contain coating excipients, such as, for example, plasticizers, talc, magnesium stearate, colorants, detackifiers, surfactants, antifoarning agents, lubricants, stabilizers, sweeteners and combinations thereof.

When formulated as a capsule, the capsule can be a hard or soft gelatin capsule, starch based capsule, a cellulose based capsule, a non-toxic digestible polymer or some combination thereof.

Soluble protein hydrolysate such as whey protein hydrolysate may be useful in liquid based pharmaceutical compositions. For example, whey protein hydrolysate can be used as a solubilizer and/or a buffering agent and/or as a viscosity modulating agent in preparing liquid based systems such as solutions and suspensions. Whey protein hydrolysate in a sufficient amount can impact the viscosity of a liquid. In an exemplary embodiment, whey protein hydrolysate in amounts of about 0.5 grams/100 ml of total volume to about 50 grams/100 ml of total volume was use to the viscosity of an aqueous based liquid composition, for example. The viscosity increased as more whey protein hydrolysate was added. Accordingly, adjustment of the amount of soluble protein hydrolysate is a parameter that can be adjusted to obtain a predetermined viscosity for a liquid.

Hydrolyzed whey protein is exemplary of a suitable soluble hydrolyzed protein for use in the practice of the invention. Whey proteins are derived from milk and are milk proteins which are soluble at pH 4.6. Membrane and/or ion exchange technology may be used to purify the whey protein. Typically, lactose components are separated physically and/or chemically from the whey protein. The protein may be hydrolyzed using chemical and/or enzymatic methods to form whey protein hydrolysate. By choice of membrane and/or ion exchange for separation and choice of the hydrolysis agent or agents to hydrolyze the whey protein to yield whey protein hydrolysate peptide fragments, the composition of the whey protein hydrolysate can be both controlled and selectively varied to yield peptide fragments with particular characteristics.

For example, selection of the hydrolysis agent determines the positions at which the whey protein is cleaved which in turn impacts the composition of the peptide fragments of the hydrolysate (e.g. the amino acid residues in the fragments). Buffering capacity of a peptide depends on the kinds of amino acids in the peptides. Accordingly, selection of a different hydrolysis agent as may yield a hydrolysate with a differing buffing capacity. Also, for example, size of the hydrolyzed fragments may impact dispersion rates and accordingly selection of hydrolysis agent can provide selectively in size of the hydrolyzed peptide fragments.

Modulation of buffering capacity and/or fragment size are representative examples and other properties relevant to how a soluble protein hydrolysate functions as an excipient may be likewise modified by selection of hydrolysis agent or agents. Selection of a mixture of proteins, selection of a separated fraction of a protein from a protein source such as a selected fraction of whey protein, for example, and/or selection of hydrolyzing agent or agents are parameters that may be adjusted to optimize the hydrolyzed whey protein for use as a pharmaceutical excipient. Additionally, multiple hydrolysis steps may be performed.

Further, once hydrolyzed, it may be desirable to select a portion of the hydrolysate for use as a excipient. For example membrane or ion exchange may be employed to select a particular portion of the protein hydrolysate for use. Although whey protein hydrolysate is typically soluble, hydrolysates of other proteins may yield a mixture of soluble and insoluble peptides. Accordingly, it may be desirable to separate soluble from insoluble peptides in some applications and/or perform additional hydrolysis steps on the insoluble portion. In some embodiments, it may be desirable to obtain two or more protein hydrolysates prepared using two or more different hydrolysis methods and/or two or more different protein sources and combine them for use as an excipient.

The amount of hydrophobic therapeutic agent to be used depends upon the dosage amount to be delivered. One skilled in the art can determine the appropriate dosage amount, depending upon the specific therapeutic agent to be delivered, the nature of the condition treated, the relative efficacy of the therapeutic agent, and other factors commonly considered. The compositions of the present invention contain a therapeutically effective amount of the therapeutic agent.

Hydrolyzed whey protein is particularly useful for preparing dosage forms of ionizable hydrophobic therapeutic agents, such as for example, ibuprofen. In some embodiments having an active such as ibuprofen, it may be desirable to add one or more amino acids, one or more salts of amino acids or a derivative of one or more amino acids or combination thereof to the composition. Arginine and lysine and their salts and/or derivatives are exemplary of suitable amino acids. Amounts of about 1% to above 80% wt/wt of amino acid, salt of amino acid or derivative of amino acid to total weight of composition may be used. Typically amino acids or their salts or derivatives may be used in an amount of about 5% wt/wt to about 20% wt/wt by weight of the total composition.

### EXAMPLES

A reference composition lacking soluble protein hydrolysate and exemplary embodiment of compositions of the invention are provided in Examples 1-6. The compositions of Examples 2-6 are representative of compositions within the scope of the invention and are provided for illustrative purposes. Amounts are given in amounts per dosage unit and are based on use of 200 mg of ibuprofen per dosage unit. This is the amount of ibuprofen in many currently available over-the-counter commercial ibuprofen products.

### Example 1

A composition similar to the compositions of the invention but lacking a soluble protein hydrolysate was prepared for comparative purposes. The compositions of this reference composition is provided in Table 1. The composition was prepared by ordered mixing. The resulting composition was formed into tablets by direct compression.

TABLE 1

| | Grade | Amount mg/du |
|---|---|---|
| Ibuprofen | USP/NF 38 | 200 |
| AerosiI ™ (Silicon dioxide) | NF 200 | 8 |
| Starch | 1500 | 80 |
| Crospovidone | low peroxide XL | 20 |
| Croscarmelose Sodium | NF | 20 |
| Avicel ™ (microcrystalline | NF ph 200 | 50 |
| cellulose) | | |
| Stearic Acid | NF Vegie | 3 |
| TOTAL | | 381 |

### Example 2

The composition of Table 2 was prepared using ordered mixing. The resulting composition was formed into tablets by direct compression.

TABLE 2

| | Grade | Amount mg/du |
|---|---|---|
| Ibuprofen | USP/NF 38 | 200 |
| Biozate ™1(soluble whey | 1 | 25 |
| protein hydrolysate) | | |
| Aerosil ™ (silicon dioxide) | NF 200 | 8 |
| Starch | 1500 | 80 |
| Crospovidone | low peroxide XL | 6.7 |
| Croscarmelose Sodium | NF | 17.5 |
| Sodium Starch Glycolate | NF | 20 |
| Avicel ™ (microcrystalline | NF ph 200 | 50 |
| cellulose) | | |
| Stearic Acid | NF Vegie | 3 |
| (TOTAL) | | 410.2 |

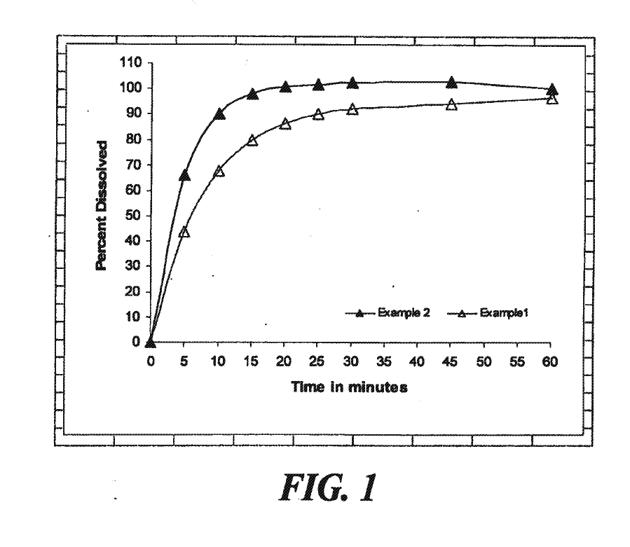

FIG. 1 is a graph showing dissolution of the composition of Example 2 as compared to the composition of Example 1 which lacked soluble whey protein hydrolysate.

### Example 3

The composition of Table 3 was prepared using ordered mixing. The resulting composition was formed into tablets by direct compression.

TABLE 3

| | Grade | Amount mg/du |
|---|---|---|
| Ibuprofen | USP/NF 38 | 200 |
| Biozate ™1 (soluble whey | 1 | 25 |
| protein hydrolysate) | | |
| Aerosil ™ (silicon | NF 200 | 8 |
| dioxide) | | |
| Milled Sucrose | | 80 |
| Crospovidone | low peroxide XL | 20 |
| Croscarmelose Sodium | NF | 20 |
| Avicel ™ (microcrystalline | NF ph 200 | 50 |
| cellulose) | | |
| Stearic Acid | NF Vegie | 3 |
| TOTAL | | 406 |

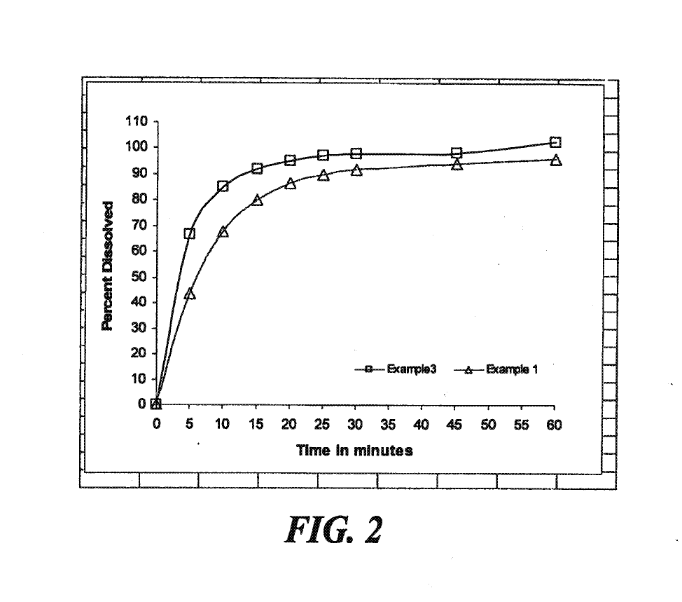

FIG. 2 is a graph showing dissolution of the composition of Example 3 as compared to the composition of Example 1 which lacked soluble whey protein hydrolysate.

### Example 4

The composition of Table 4 was prepared using ordered mixing and tableted using direct composition methods.

TABLE 4

| | Grade | Amount mg/du |
|---|---|---|
| Ibuprofen | USP/NF 38 | 200 |
| Biozate ™1 (soluble whey | 1 | 25 |
| protein hydrolysate) | | |
| Aerosil ™ (silicon dioxide) | NF 200 | 8 |
| Starch | 1500 | 80 |
| Crospovidone | low peroxide XL | 20 |
| Croscarmelose Sodium | NF | 20 |
| Avicel ™ (microcrystalline | NF ph 200 | 50 |
| cellulose) | | |
| Stearic Acid | NF Vegie | 3 |
| TOTAL | | 410.2 |

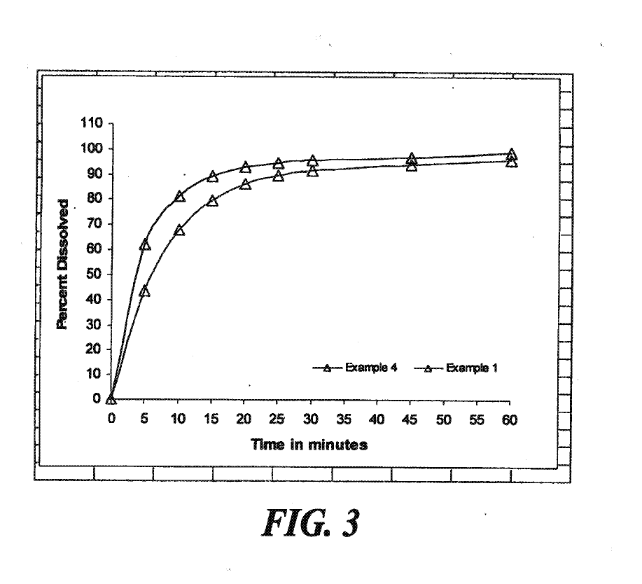

FIG. 3 is a graph showing the dissolution of the composition of Example 4 as compared to the composition of Example 1 lacked soluble whey protein hydrolysate.

### Example 5

The composition of Table 5 was prepared using ordered mixing. The resulting composition was tableted using direct compression.

TABLE 5

| | Grade | Amount mg/du |
|---|---|---|
| Ibuprofen | USP/NF 38 | 200 |
| Biozate ™1 (soluble whey | 1 | 25 |
| protein hydrolysate) | | |
| Aerosil ™ (silicon dioxide) | NF 200 | 8 |
| Lysine HCL | | 80 |
| Crospovidone | low peroxide XL | 20 |
| Croscarmelose Sodium | NF | 20 |
| Avicel ™ (microcrystalline | NF ph 200 | 50 |
| cellulose) | | |
| Stearic Acid | NF Vegie | 3 |
| TOTAL | | 406 |

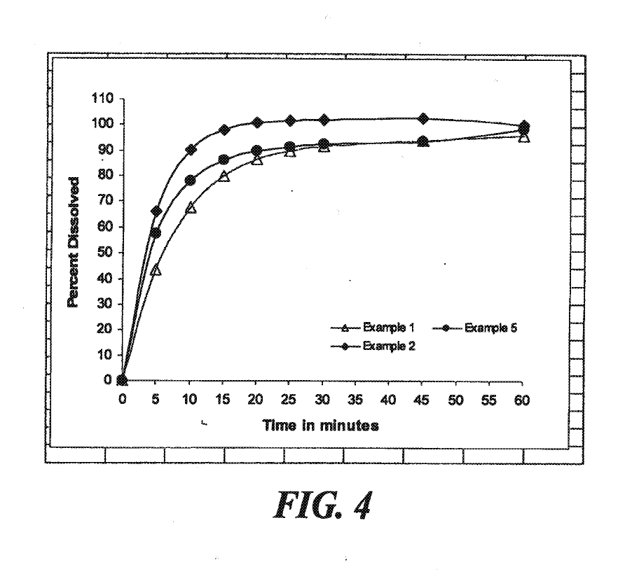

FIG. 4 is a graph showing the dissolution of the composition of Example 5 as compared to the composition of Example 1 and the composition of Example 2. The composition of Example 1 lacked soluble whey protein hydrolysate and the amino acid salt. The composition of Example 2 has soluble whey protein hydrolysate but does not include an amino acid.

### Example 6

The composition of Table 6 was prepared using ordered mixing. The resulting composition was formed into tablets using direct compression.

TABLE 6

| | Grade | Amount mg/du |
|---|---|---|
| Ibuprofen | USP/NF 38 | 200 |
| Biozate ™1 (whey protein | 5 | 25 |
| hydrolysate) | | |
| Aerosil ™ (silicon dioxide) | NF 200 | 4 |
| Croscarmelose Sodium | NF | 20 |
| Avicel ™ (microcrystalline | NF ph 200 | 110 |
| cellulose) | | |
| Magnesium Stearate | NF | 2 |
| TOTAL | | 361 |

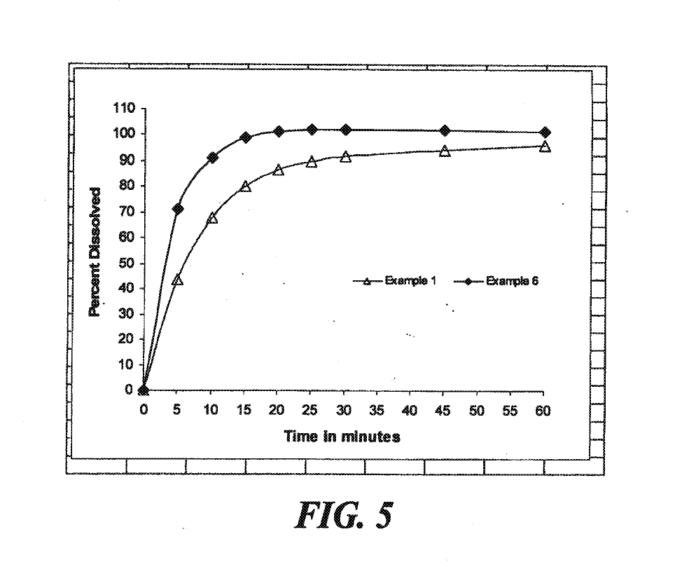

FIG. 5 is a graph showing the dissolution of the composition of Example 6 as compared to the composition of Example 1 which lacked whey protein hydrolysate.

Although the foregoing invention has been described in some detail by way of illustrations and examples for purposes of clarity of understanding, it will be obvious that certain changes and modifications, may be practiced within the scope of the claims. Modifications of the above-described modes of producing the invention that are obvious to persons of skill in the art are intended to be included within the scope of the following claims.

1. A pharmaceutical composition comprising an effective amount of ibuprofen and about 0.01 up to about 20% wt/wt whey protein hydrolysate to total weight of the composition.

2-4. (canceled)

5. The pharmaceutical composition of claim 1, wherein the composition is a dosage form selected from the group consisting of a mini-capsule, a capsule, a tablet, a troche, a lozenge, a minitablet, a suspension, an ovule, a suppository, a wafer, a chewable tablet, an effervescent tablet, a caplet, a buccal or sublingual solid, a granulation, a microsphere, a foam, a film, a sprinkle, a pellet, a bead, a pill, a powder, a triturate, a platelet, a strip, a sachet, a lyophilized cake and combinations thereof.

6-10. (canceled)

11. The pharmaceutical composition of claim 1, wherein the ibuprofen comprises at least one of a neutral species, an ionized species, and salt.

12-14. (canceled)

15. The pharmaceutical composition of claim 1, further comprising at least one amino acid, amino acid salt, or derivative thereof.

16-21. (canceled)

22. The pharmaceutical composition of claim 1, wherein the pharmaceutical composition is prepared by granulation.

23. The pharmaceutical composition of claim 1, wherein the pharmaceutical composition is prepared by mixing.

24. The pharmaceutical composition of claim 1, wherein the composition comprises about 8% to about 18% wt/wt whey protein hydrolysate to weight of whey protein hydrolysate plus ibuprofen.

25. The pharmaceutical composition of claim 1, further comprising at least one amino acid amino acid salt or derivative thereof derivative thereof.

26. The composition of claim 1, further comprising at least one other excipient.

27. A method of preparing a pharmaceutical composition comprising:
providing a whey protein hydrolysate,
providing an effective amount of ibuprofen; and
combining the whey protein hydrolysate and effective amount ibuprofen.

28. The method of claim 27, wherein combining the whey protein hydrolysate and effective amount of ibuprofen is selected from the group consisting of dry mixing, solvent mixing, agglomerating, air suspension chilling, air suspension drying, balling, coacervations, coating, compressing, cryopelletization, encapsulation, extrusion, wet granulation, dry granulation, homogenization, inclusion complexation, lyophilization, melting, microencapsulation, mixing, molding, pan coating, precipitation, solvent dehydration, sonication spheronization, spray chilling, spray congealing, spray drying, melting and cooling with recrystallization and combinations thereof.

29. The method of claim 27, wherein the whey protein hydrolysate and effective amount of ibuprofen are combined by granulation, further comprising compacting the granulation.

30. The method of claim 29, wherein the granulation is compacted up to about 20%.

31. The method of claim 29, wherein the granulation is compacted greater than about 20%.

32. The method of claim 27, further comprising, preparing the composition in an dosage form selected from the group consisting of a mini-capsule, a capsule, a tablet, a troche, a lozenge, a minitablet, a suspension, an ovule, a suppository, a wafer, a chewable tablet, an effervescent tablet, a caplet, a buccal or sublingual solid, a granulation, a microsphere, a foam, a film, a sprinkle, a pellet, a bead, a pill, a powder, a triturate, a platelet, a strip, a sachet lyophilized cake and combinations thereof.

33. The method of claim 32, wherein the dosage form is a tablet further comprising coating the tablet.

34. The method of claim 33, wherein the coating is selected from the group consisting of film coat, modified film coat, sugar coat, compression coat or laminates.

35-37. (canceled)
